(19) United States
(12) Patent Application Publication
(10) Pub. No.: US 2020/0262948 A1
(43) Pub. Date: Aug. 20, 2020

(54) Functionalized Polyvinyl Alcohol and Products Formed Therewith

(71) Applicant: UNIVERSITY OF SOUTH CAROLINA, Columbia, SC (US)

(72) Inventors: R. MICHAEL GOWER, Columbia, SC (US); CHRISTOPHER ISELY, Cottage Grove, MN (US)

(21) Appl. No.: 16/707,423

(22) Filed: Dec. 9, 2019

Related U.S. Application Data
(60) Provisional application No. 62/805,458, filed on Feb. 14, 2019.
(60) Provisional application No. 62/832,418, filed on Apr. 11, 2019.

(51) Int. Cl. C08F 116/06; C08J 5/18; A61K 9/16; A61K 31/05; C08F 8/34; G01N 33/58; G01N 21/64

(52) U.S. Cl. CPC C08F 116/06; C08J 5/18; A61K 9/1682; A61K 9/1635; A61K 9/1647; G01N 2021/6439; C08F 8/34; G01N 33/582; G01N 21/6428; C08J 2329/04; A61K 31/05

(57) ABSTRACT

Derivatized polyvinyl alcohols and methods for forming derivatized polyvinyl alcohols are described. Methods include incubating a polyvinyl alcohol with a derivatizing agent that includes an isothiocyanate group, upon which the isothiocyanate can spontaneously react with alcohol groups of the polyvinyl alcohol. Functionalization can include addition of detectable labels and/or active groups the polyvinyl alcohol. Derivatized materials can be used in forming products, e.g., particles, films, fibers, that include the added functionality at a surface.

### CROSS REFERENCE TO RELATED APPLICATION

This application claims filing benefit of U.S. Provisional Patent Application Ser. No. 62/805,458, entitled “Development of Microparticles for Controlled Release of Resveratrol to Adipose Tissue and the Impact of Drug Loading on Particle Morphology and Drug Release,” having a filing date of Feb. 14, 2019; and claims filing benefit of U.S. Provisional Patent Application Ser. No. 62/832,418, entitled “Functionalized Polyvinyl Alcohol and Products Formed Therewith,” having a filing date of Apr. 11, 2019, both of which are incorporated herein by reference for all purposes.

### BACKGROUND

Polyvinyl alcohol (PVA) is a non-toxic water-soluble synthetic polymer with many useful characteristics including film forming, emulsifying, adhesiveness, high tensile strength, flexibility, and excellent barrier properties, as well as being resistant to organic materials (e.g., oil, grease, solvents). As such, it is widely used in a variety of different industries, such as paper making (as a binder), textiles (as a sizing agent), in formation of water-soluble products (e.g., packaging, backing sheets), and in a variety of different coating applications (e.g., CO₂ barrier films in plastic bottles, chemical barrier films in protective gear, etc.). Due to its non-toxicity and biodegradability, it has been particularly attractive for use in development of drug delivery agents, for instance, as an emulsifier in the production of drug-carrying particles.

It is often desirable to modify the surface characteristics of products formed with PVA, for instance, through inclusion of dyes or other labeling agents or inclusion of active agents to provide specific binding characteristics, pH characteristics, charge characteristics, etc. Unfortunately, carrying out a separate surface modification step following formation of a product adds to productions costs and may detrimentally affect other desired qualities of the product. Similarly, inclusion of an additional or substituted material in the formation process itself in order to provide a sought-after surface characteristic may detrimentally affect other qualities of the product. For example, in order to change the surface chemistry of a drug delivery particle, standard current practice is to utilize a different emulsifier other than PVA. However, in doing so, unintended changes to the system can take place, such as undesirable modification of the drug loading amount or system release rate.

What is needed in the art is functionalized PVA that can be utilized in forming PVA-containing products so as to provide the functionalization chemistry to the product, e.g., the product surface. A simple, low-cost method for forming a functionalized PVA that maintains the desirable qualities of the starting PVA with the additional benefits provided by the functionalization group would be of great benefit.

### SUMMARY

According to one embodiment, disclosed is a functionalized PVA having the following structure:

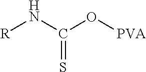

in which PVA comprises a polyvinyl alcohol and R comprises a functional group of choice. For instance, R can include a labeling group, e.g., a visually detectable group such as a fluorescein group, a rhodamine group, or the like. The R group can provide any desired functionality to the polymer, e.g., color, binding, charge, etc. In one embodiment, the R group can provide a biological activity, e.g., cell surface binding, enzymatic activity, immune system modulation, etc.

Also disclosed are methods for forming the functionalized PVA. The methods can include dissolving PVA in solution with a derivatizing agent that includes an isothiocyanate group and a functional group and incubating for a period of time, e.g., in dimethyl sulfoxide (DMSO). Upon incubation at reaction conditions, e.g., at room temperature for a period of about 30 minutes or more, the reaction of the isothiocyanate group with alcohol groups of the PVA can be more favorable than reaction of the functional group with the vinyl groups along the backbone of the PVA and the functionalized PVA can form. The reaction mechanism is understood to be as follows:

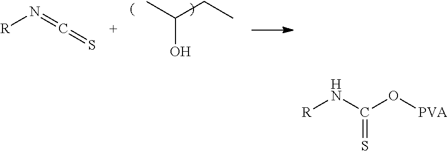

Also disclosed are products that incorporate the functionalized PVA, e.g., a film, fiber, or particle that incorporates the functionalized PVA, for instance, at a surface of the product. In one embodiment, disclosed is a biocompatible particle formed according to an oil-in-water solvent evaporation method in which the functionalized PVA is employed as an emulsifier. The product particle can include the functionalized PVA at a surface of the particle. Derivatized particles can, in one embodiment, be utilized in drug delivery, e.g., a poly(lactide-co-glycolide) (PLG)-based particle including a desirable surface chemistry and a bioactive agent carried by the biodegradable particle.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 presents confocal imaging of particles made with fluorescein labeled-PVA (FITC-PVA). Fluorescent images of particles made with oil phases consisting of 6% PLG in (A) DCM (B) 1:9 ethanol:DCM, (C) 1:9 ethanol:DCM containing 4 mg/mL resveratrol, and (D) 1:3 ethanol:DCM containing 10 mg/mL resveratrol. FITC signal intensity was quantified using ImageJ and is depicted in panel (E) as mean±standard deviation for 27 particles per group. Scale bar is 20 μm.

FIG. 2 illustrates flow cytometry of resveratrol particles made with FITC-PVA. (A) Histograms depicting FITC fluorescence intensity of particles made with oil phases consisting of 6% PLG in DCM (blue), 1:9 ethanol:DCM (red), and 1:9 ethanol:DCM containing 4 mg/mL resveratrol (green). (B) Mean fluorescence intensity (MFI) and standard deviation of data from panel (A). 600 particles were analyzed for each group.

Repeat use of reference characters in the present specification and drawings is intended to represent the same or analogous features or elements of the present invention.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment may be used in another embodiment to yield a still further embodiment.

In general, disclosed is a method for forming a functionalized PVA using mild chemistry that maintains desirable characteristics of the PVA with the addition of a beneficial functional group to the polymer. Also disclosed are products that can be formed incorporating the functionalized PVA. Beneficially, the methods provide a route to tune the chemistry of PVA in a highly controlled fashion so as to provide a predetermined derivatization concentration on the polymer and, thereby, can retain desired characteristics of the starting, non-functionalized PVA. Products formed with the functionalized PVA can incorporate the chemistry provided by the derivatization agent as pendant groups to the polymer backbone without loss of desirable PVA qualities. The ability to tune PVA chemistry in a controlled manner, using a simple, mild chemical reaction, can allow for a PVA-containing product to include additional characteristics, for example: detectable labeling, targeted adhesion, modified pH, modified charge characteristics, color, etc., without detrimental effect to the product.

In one particular embodiment, disclosed methods can be utilized to form drug delivery particles according to the well-known oil-in-water solvent evaporation method in which the functionalized PVA is employed as an emulsifier. As such, disclosed techniques can provide for modification of a drug carrier's surface chemistry through addition of the functionalization chemistry, enabling new drug delivery capabilities, and avoiding undesirable consequences to the delivery profile.

The PVA functionalization methods utilize a derivatization agent that includes a functional group of choice and an isothiocyanate group for reaction with an alcohol of the PVA. More specifically, during a derivatization reaction, the isothiocyanate group of the derivatization agent can react with an alcohol on a PVA and to form a monothiourethane linkage. A general reaction mechanism for disclosed derivatizations is understood to be as follows:

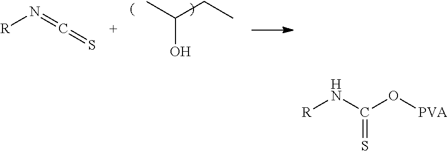

in which R includes a functional group of choice and in which the reaction between the isothiocyanate group and an alcohol of the PVA is more favorable than a reaction between the functional group and the vinyl groups along the backbone of the PVA at the reaction conditions.

In general, the functionalization reaction can be carried out under mild conditions (e.g., room temperature, atmospheric pressure). In addition, the derivatization level of the PVA can be easily controlled through control of the concentration of derivatization agent used during the functionalization reaction.

For example, in one embodiment, a generalized reaction mechanism for the derivatization reaction can be as follows:

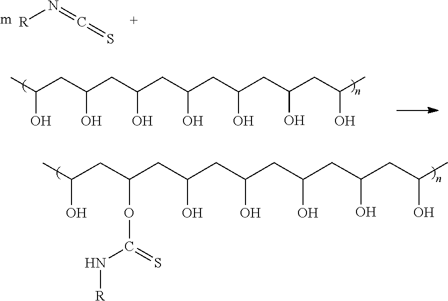

in which m, n are 1 or greater, i.e., the PVA can be functionalized on a single OH group of the polymer (m=n) or on multiple or all OH groups of the polymer (m>n). For example, the molar percentage of functionalized OH groups of the PVA can be from about 0.1 mol % to 100 mol %, from about 0.2 mol % to about 90 mol %, from about 0.3 mol % to about 80 mol %, from about 0.5 mol % to about 75 mol %, from about 1 mol % to about 50 mol %, or from about 2 mol % to about 10 mol % in some embodiments. In particular embodiments, PVA can be functionalized with the derivatization agent incorporated on the polymer in an amount of about 0.1%, 1%, 10% or more per mole of OH group present on the polymer.

Thus, through control of the concentration of the derivatization agent in the reactant mixture, the ultimate concentration of the added functionalization pendant to the PVA backbone can likewise be controlled.

There are no particular limitations on the size or type of PVA that can be functionalized according to disclosed methods or the source of the PVA. For instance, the PVA can be prepared according to standard practice by the hydrolysis of a homopolymer or copolymer of polyvinyl acetate. By way of example; polyvinyl alcohols available from Sigma-Aldrich can be utilized.

In one embodiment, a PVA can have about 85% or greater hydrolysis of the acetate groups, or about 95% or greater hydrolysis of the acetate groups in some embodiments. However, lower or higher hydrolysis levels are also encompassed. For instance, the PVA can be fully hydrolyzed in some embodiments.

In addition, the size of the PVA is not limited. For instance, the weight average molecular weight of PVA may be about 5,000 or greater, for instance, from about 5,000 to about 500,000, from about 10,000 to about 200,000, or from about 12,000 to about 50,000 in some embodiments.

Both PVA homopolymers and copolymers can be derivatized according to disclosed methods. The utilization of a homopolymer or a copolymer can generally depend upon the desired application for the functionalized PVA. For instance, in forming a film, it may be preferred to form a functionalized PVA homopolymer, as copolymers of polyvinyl acetate have been known to provide less than optimum film properties. Similarly, when intended for use as an emulsifier, it may be preferred in some embodiments to utilize a PVA homopolymer. As indicated, however, the derivatization process is not limited to any particular PVA homopolymer or copolymer.

The derivatizing agent can include (or be modified to include) an isothiocyanate group for reaction with an alcohol of the PVA chain in conjunction with a functional group of choice. The functional group can provide one or more desired characteristics to the PVA and/or to the product to be formed with the PVA.

In one embodiment, a functional group can provide a detectable label to the PVA. For example, a functional group can incorporate an optically detectable label, e.g., a fluorescent or phosphorescent label. Compounds including both an optically detectable label and an isothiocyanate group as are available in the market can be utilized in one embodiment. For instance, isothiocyanate-containing labeling agents such as fluorescein isothiocyanate (FITC), tetramethylrhodamine isothiocyanate, or rhodamine B isothiocyanate (RITC) as are readily available in the market can be utilized.

Detectable labels are not limited to such materials however, and in other embodiments, a desired labeling agent may be functionalized to include an isothiocyanate group and this product utilized as a derivatizing agent for a PVA. For instance, a derivatization agent can be formed to include an optically detectable label that exhibits a relatively long emission lifetime, so that the label emits its signal well after any short-lived background signals dissipate. In addition, an optically detectable label may have a relatively large Stokes shift, so as to improve separation of the excitation wavelength of the label to remain from its emission wavelengths. One type of fluorescent compound that has both a relatively long emission lifetime and relatively large Stokes shift are lanthanide chelates, such as chelates of samarium (Sm(III)), dysprosium (DOHA europium (Eu(III)), and terbium (Tb(III)). Another type of fluorescent compound that has both a relatively long emission lifetime and relatively large Stokes shift are transition metal chelates, such as chelates of ruthenium (Ru(II)), osmium (Os(II)), and rhenium (Re(I)). These, as well as other detectable labels as are known in the art, are encompassed herein.

Of course, a functional group is not limited to detectable labels, and a functional group can provide any characteristic as desired to PVA including, without limitation, color, adhesion, specific bonding, charge characteristics, pH characteristics, biological activity (e.g., enzymatic activity, immune system activity, etc.). For instance, a bioactive functional group such as a sugar, a polypeptide, a glycoprotein, a bioactive small molecular, or the like, can be functionalized to include, an isothiocyanate group, and a PVA can then be derivatized to include the bioactive functional group. In certain embodiments, a PVA can include more than one, such as 2, 3, 4, or 5 different functional groups. For instance, PVA can be derivatized to include a detectable label in conjunction with a binding agent. Moreover, multiple functional groups can be provided in a single derivatizing agent or, alternatively, PVA can be derivatized with multiple different derivatization agents, with different agents differing according to functional group(s) carried by the agents.

In one embodiment, the functional group of the functionalized PVA can include a specific binding agent, for instance as may be utilized in targeting a PVA-containing drug carrier to a delivery site. In certain embodiments, the specific binding agent can be an antibody or binding fragment thereof, a functional nucleic acid, peptide, peptide mimetic, affibody, diabody, glycoprotein, glycopeptide, proteoglycan, avimer, nanobody, adnectin, small molecule (e.g., Mw less than 2000 g/mol), carbohydrate, lipid, spiegelmer, and so forth. In certain embodiments, a specific binding agent can specifically bind more than one target.

PVA carrying a bioactive specific binding agent can bind a healthy cell, a cancer cell, extracellular matrix, a tissue, a protein, a carbohydrate, a glycoprotein, a proteoglycan, a lipid, a nucleic acid, an amino acid, or any other suitable target. In one embodiment, the specific binding agent can bind a cell and trigger cellular internalization of the drug following release from the drug carrier that includes the functionalized PVA.

A functional group can likewise trigger other biological activities, e.g., transcellular transport, endosome escape, etc., as well as activities that can be useful in both biological and non-biological applications, e.g., surface binding, adsorption, conjugation, interactions, complexing, shielding, activation, etc.

Methods and chemistries for derivatizing a compound that includes the desired functional group to include an isothiocyanate are known in the art. For instance, phenyl isothiocyanate derivatives can be formed as described in U.S. Pat. Nos. 3,231,596 and 4,228,165, which are incorporated herein by reference in their entirety. By way of example, in one embodiment, an isothiocyanate-containing derivatizing agent can be formed through reaction of an amine-containing derivative (e.g., an aromatic amine derivative) with carbon disulfide in the presence of a base in a nitrogen-containing polar solvent.

Any isothiocyanate-containing derivatizing agent can be utilized and can be formed (as necessary) according to any suitable reaction chemistry as is known. A large number of isothiocyanate reactants are readily available in the art (e.g., alkyl isothiocyanates, alkoxy isothiocyanates, aromatic isothiocyanates, acetyl isothiocyanates, cycloalkyl isothiocyanates, etc.), that can readily be functionalized according to known chemistries to include a functional group of interest for use as an isothiocyanate-containing derivatizing agent in disclosed methods.

R-groups of interest can include, but are not limited to, proteins, carbohydrates, lipids, glycosides, indoles, peptides, polyphenols, nucleic acids, glycoproteins, glycosaminoglycans, lipoproteins, and the like. Due to the utility of isothiocyanate chemistry in industry, many R-groups of interest containing an isothiocyanate group are commercially available. In some embodiments, however, it may be preferable to form a particular isothiocyanate-containing derivatizing agent. One exemplary embodiment for forming a functionalized isothiocyanate-containing derivatizing agent can include a reaction between a primary amine of an R-group and carbon disulfide in aqueous ammonia. This results in precipitation of an ammonium dithiocarbamate salt, which can then be treated with lead nitrate to yield the corresponding isothiocyanate. In another embodiment, a dithiocarbamate salt can be treated with tosyl chloride to yield the corresponding isothiocyanate.

To form the functionalized PVA, a derivatizing agent that carries the functional group(s) of interest in conjunction with an isothiocyanate group can be combined with PVA in a suitable solvent under reaction conditions in which the reaction between the isothiocyanate group and an alcohol of the PVA will be more favorable than a reaction between the functional group and the PVA. For instance, when considering a derivatizing agent that carries a detectable label, the derivatizing agent may include the isothiocyanate as the only group of the agent that exhibits reactivity to PVA. In other embodiments, a derivatizing agent can include a functional group that is either non-reactive to PVA or that is non-reactive to PVA under the conditions of the PVA derivatization reaction.

During the derivatization reaction, the isothiocyanate group on the derivatizing agent can spontaneously react with an alcohol group on PVA to form a monothiourethane linkage. In some embodiments, the reaction can be carried out at mild conditions, e.g., stirring the two reactants together at room temperature in a suitable solvent (e.g., DMSO).

Following a period of time, e.g., about 30 minutes or more, for instance from about 1 hour to about 24 hours, or from about 2 hours to about 10 hours in some embodiments, the newly functionalized PVA can then be recovered using, e.g., lyophilization. As discussed previously, the extent of functionalization of the PVA can be controlled by the relative proportions of PVA alcohol content and the derivatization agent.

Functionalized PVA as described can be utilized in any application as is known for the similar, but non-functionalized PVA. By way of example, in one embodiment, a modified polyvinyl alcohol can be used in drug delivery as an emulsifier in an oil-in-water emulsion/solvent evaporation method in which a functionalized PVA can be incorporated as an emulsifier in formation of biodegradable particles as depots for controlled release of an active agent from the particles.

An oil-in-water emulsion/solvent evaporation particle formation method generally includes combination of an organic phase, which includes the matrix polymer of the particle in solution in combination with the bioactive agent that is to be carried/delivered by the particle, with an aqueous phase, which includes the functionalized PVA as an emulsifier. In those embodiments in which the bioactive agent to be carried/delivered from the particles is not soluble in the organic solvent, a double emulsion process can be carried out according to standard processes.

Polymers as may be utilized in forming the polymeric matrix of the particles can encompass any biocompatible, biodegradable polymer as is known in the art including, without limitation, poly(ethylene oxide)-poly(propylene oxide)-poly(ethylene oxide) (PEO-PPO-PEO) copolymer, poly(lactide-co-glycolide) (PLG), polycaprolactone (PCL), poly(lactic acid) (PLA), poly(glycolic acid) (PGA), polyethylene glycol, and polysorbate, or any combination of biocompatible, biodegradable polymers. Any suitable organic solvent for the polymer and, optionally, also the bioactive agent can be utilized.

Upon combination under agitation, an emulsion is formed including the oil phase dispersed in the aqueous continuous phase. The PVA emulsifier serves to stabilize the dispersed organic droplets at the water/oil interface and inhibit coalescent of the droplets. Once the emulsion is formed, the solvent can be removed by, e.g., evaporation and/or solvent extraction. The conditions of solvent removal (reduced pressure, temperature, etc.) encourage loss of the organic solvent from the droplets via leaching, diffusion, etc. and solidification of the polymer of particles. The particles will include an amount of the functionalized PVA at the surface and the bioactive agent held by the polymeric matrix of the particles.

Through control of formation conditions, the size and porosity of the resulting particles can be controlled, as is known. For instance, for in vivo delivery, limiting particle size to a range above 5 microns can be useful so as to inhibit phagocytic clearance and produce drug release profiles with a duration expected to sustain the bioactive agent carried by the particles at desired levels.

Other PVA-containing products as can be formed using the functionalized PVA can include, without limitation, films, coatings, fibers, sizing agents, binders, etc. For instance, a functionalized PVA-containing composition can be extruded or solution cast to form a fiber or a film that incorporates the added functionality of the functionalized PVA, e.g., at the surface of the film.

The present disclosure may be better understood with reference to the Examples set forth below.

### Example 1

75:25 poly (D,L-lactide-co-glycolide ((PLG) with a lauryl ester end group and an inherent viscosity of 0.79 dL/g was purchased from Evonik. Dichloromethane, resveratrol, PVA (Mw 13,000-23,000, 87-89% hydrolyzed), and FITC were purchased from Sigma. 200 proof ethanol was purchased from Decon Laboratories. Dimethyl sulfoxide (DMSO) was purchased from Fisher. Ultrapure water was obtained from a Thermo Scientific Barnstead Nanopure system.

PVA was dissolved in DMSO at 50 mg/mL. FITC and dry KOH were added to the solution at concentrations of 3.5 mg/mL and 0.5 mg/mL, respectively. This solution was stirred at room temperature for 5 hours. The solution was then dialyzed using dialysis tubing (3.5 kDa MWCO) in 3 L of ultrapure water for 3 days, replacing the water twice daily. The FITC labeled PVA (FITC-PVA) was collected, frozen, and lyophilized. This FITC-PVA was then used to fabricate PLG particles.

PLG particles were prepared using a single oil-in-water emulsification/solvent evaporation method. Briefly, PLG was dissolved in dichloromethane (DCM) at 6% (weight/weight) and resveratrol was dissolved in 100% ethanol at 40 mg/mL. The organic phase, which consisted of varying volume ratios of ethanol and DCM, was added to an aqueous solution of FITC-PVA at 1 or 2% (weight/volume) in a 1:7 volume ratio and homogenized using a Kinematica PT3100D homogenizer. The emulsion was then added to an aqueous solution of FITC-PVA and stirred for 5 hours, allowing the DCM and ethanol to evaporate and the particles to harden. The volume of FITC-PVA solution in the solvent evaporation beaker was 5 times greater than the emulsion mixture. The particles were then passed through a 40 μm filter, collected via centrifugation, and washed 4 times in ultrapure water. Particles were frozen at −20° C. and lyophilized overnight. Particles were then stored protected from light, under vacuum, at room temperature.

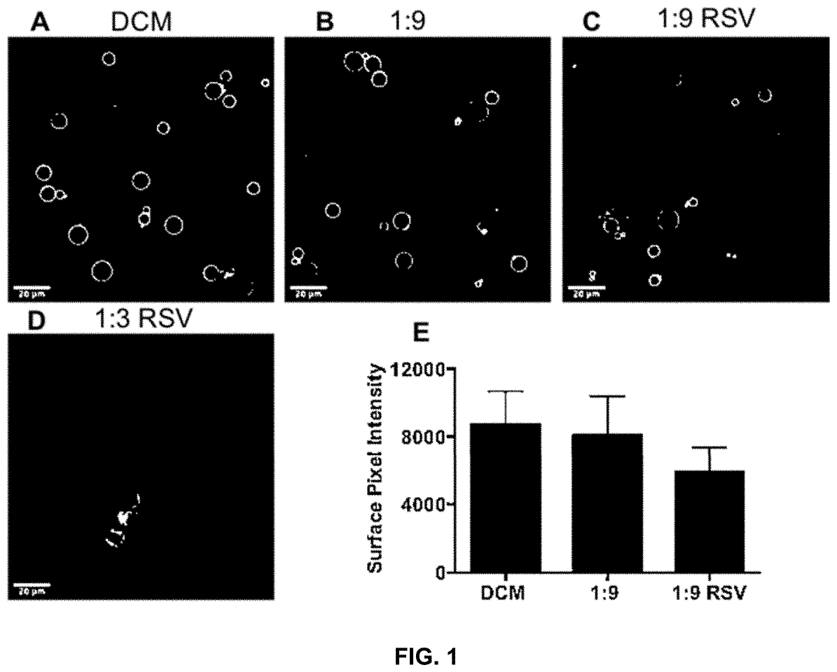

Particles were suspended in a 50:50 mixture of ultrapure water and anti-fade mounting media, pipetted on a slide, mounted with a coverslip, and dried overnight. Images were acquired on a Zeiss 700 confocal microscope using the 488 nm laser (FIG. 1). Particles were analyzed for fluorescence intensity using ImageJ as follows. A line was drawn through each particle and the maximum intensity of FITC on the surface was recorded using the plot profile function. 27 particles were analyzed for each condition. Data is mean±standard deviation of the measured surface pixel intensity.

The confocal microscopy of the spherical particles indicated that FITC-PVA was present on the surface of the particle and evenly distributed (FIG. 1A, FIG. 1B, FIG. 1C). This was the case whether or not resveratrol was incorporated into the particle; however, particles made with a high resveratrol loading (65 μg/mg) exhibited an altered FITC signal that suggested PVA was not evenly distributed over the particle's surface (FIG. 1C). While not strongly apparent in the confocal images by eye, image analysis revealed that the FITC signal at the surface of the spherical particles was decreased when resveratrol was encapsulated into the particle (FIG. 1E).

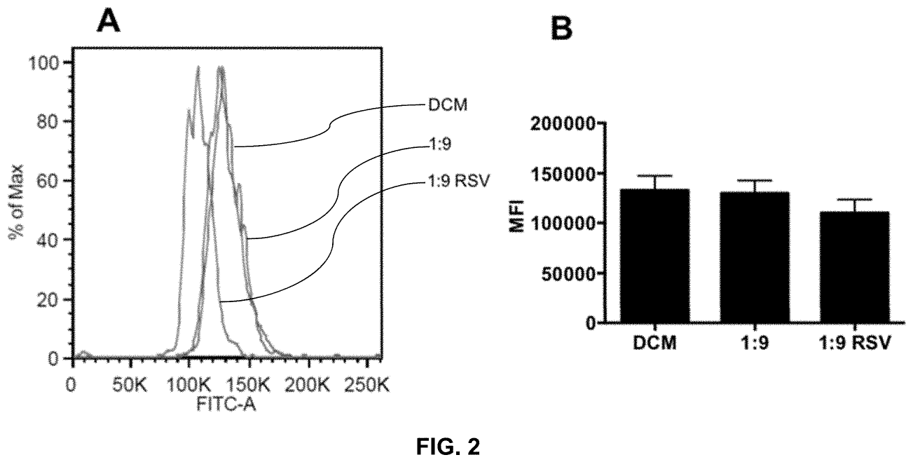

To confirm the confocal data, flow cytometry of the particles was conducted. Particles were suspended in ultrapure water and analyzed with a BD FACS Aria flow cytometer for FITC fluorescent intensity. Approximately 600 particles were analyzed for each condition. Data was analyzed using FlowJo (Treestar). Results are presented in FIG. 2.

Congruent with the confocal data, the FITC signal was decreased on resveratrol particles. Taken together, the data suggests that resveratrol incorporation into the particle decreased the amount of PVA that associated with the particle's surface, suggesting that resveratrol accumulated at the particle surface.

### Example 2

PVA (Mw 13,000-23,000, 87-89% hydrolyzed) was dissolved in DMSO at 50 mg/m L. FITC and dry KOH were added to the solution at various concentrations to provide three samples, the samples including the FITC at molar concentration of 0.1%, 1%, or 10%, respectively, by mole of OH present on the PVA. The solutions were stirred at room temperature for 5 hours. The solutions were then dialyzed using dialysis tubing (3.5 kDa MWCO) in 3 L of ultrapure water for 3 days, replacing the water twice daily. The FITC labeled PVA (FITC-PVA) was collected, frozen, and lyophilized. Visual examination of the resulting materials demonstrated the increased loading of the FITC on the polymer, with increasing levels of add-on exhibiting an increase in yellow color for the polymer.

While certain embodiments of the disclosed subject matter have been described using specific terms, such description is for illustrative purposes only, and it is to be understood that changes and variations may be made without departing from the spirit or scope of the subject matter.

What is claimed is:

1. A functionalized polyvinyl alcohol having the following structure: 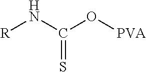
in which PVA comprises a polyvinyl alcohol and R comprises a functional group.

2. The functionalized polyvinyl alcohol of claim 1, wherein R comprises a detectable label.

3. The functionalized polyvinyl alcohol of claim 2, wherein the detectable label comprises a fluorescent detectable label.

4. The functionalized polyvinyl alcohol of claim 1, wherein R comprises a specific binding agent.

5. The functionalized polyvinyl alcohol of claim 1, wherein R comprises a bioactive functional group.

6. The functionalized polyvinyl alcohol of claim 1, comprising multiple different functional groups.

7. A film or a fiber comprising the functionalized polyvinyl alcohol of claim 1.

8. A particle comprising the functionalized polyvinyl alcohol of claim 1.

9. The particle of claim 8, the particle comprising the functionalized polyvinyl alcohol at a surface of the particle, the particle further comprising a polymeric matrix comprising a biocompatible and biodegradable polymer and a bioactive agent carried by the polymeric matrix.

10. A method for forming a functionalized polyvinyl alcohol comprising:
combining a polyvinyl alcohol and a derivatizing agent in a solution, the derivatizing agent comprising an isothiocyanate group; and
maintaining the solution under conditions to encourage reaction of the isothiocyanate group with an alcohol of the polyvinyl alcohol.

11. The method of claim 10, the conditions comprising room temperature and atmospheric pressure.

12. The method of claim 10, wherein the polyvinyl alcohol is a polyvinyl alcohol homopolymer.

13. The method of claim 10, the derivatizing agent further comprising a detectable label.

14. The method of claim 10, the derivatizing agent further comprising a bioactive functional group.

15. The method of claim 10, further comprising forming the derivatizing agent.

16. A method for forming a particle comprising:
combining an organic phase and an aqueous phase to form an emulsion, the organic phase comprising a biocompatible and biodegradable polymer dissolved in a solvent, the aqueous phase comprising a functionalized polyvinyl alcohol having the following structure: 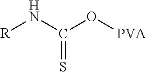
in which PVA comprises a polyvinyl alcohol and R comprises a functional group, the emulsion comprising droplets of the organic phase dispersed in the aqueous phase; and
removing the solvent from the droplets, wherein upon removal of the solvent the biocompatible and biodegradable polymer solidifies to form a polymeric matrix of the particles, the particles comprising the functionalized polyvinyl alcohol at a surface of the particles.

17. The method of claim 16, the organic phase further comprising a bioactive agent, wherein upon solidification of the particles, the bioactive agent is held by the polymeric matrix.

18. The method of claim 16, the biocompatible and biodegradable polymer comprising poly(ethylene oxide)-poly(propylene oxide)-poly(ethylene oxide) copolymer, poly(lactide-co-glycolide), polycaprolactone, poly(lactic acid), poly(glycolic acid), polyethylene glycol, polysorbate, or any combination thereof.

19. The method of claim 18, the biocompatible and biodegradable polymer comprising poly(lactide-co-glycolide).

20. The method of claim 16, the functional group comprising a detectable label.
